(19) United States
(12) Patent Application Publication
(10) Pub. No.: US 2016/0338591 A1
(43) Pub. Date: Nov. 24, 2016

(54) SYSTEMS AND METHODS FOR MITIGATING TISSUE BREAKDOWN

(71) Applicant: Hill-Rom Services, Inc., Batesville, IN (US)

(72) Inventors: Charles A. Lachenbruch, Batesville, IN (US); Rachel L. Williamson, Batesville, IN (US)

(73) Assignee: Hill-Rom Services, Inc., Batesville, IN (US)

(21) Appl. No.: 15/149,438

(22) Filed: May 9, 2016

Related U.S. Application Data
(60) Provisional application No. 62/164,811, filed on May 21, 2015.

(51) Int. Cl. A61B 5/00; A61B 5/01

(52) U.S. Cl. CPC A61B 5/0059; A61B 5/447; A61B 5/445; A61B 5/002; A61B 5/7405; A61B 5/7455; A61B 5/742; A61B 5/01

(57) ABSTRACT

A method for tracking a change in tissue status includes receiving at least some demographic information regarding an individual being monitored, receiving information regarding a first tissue status for the individual, comparing the received information with a threshold tissue status selected at least in part based on the at least some demographic information regarding the individual, and providing an alert indicative of a change in tissue status in response to determining that the received information exceeds the threshold tissue status. A system for implementing the methods of various embodiments are also provided.

### CROSS-REFERENCE TO RELATED APPLICATIONS

The present application claims priority to U.S. Provisional Patent Application Ser. No. 62/164,811 filed May 21, 2015 and entitled “SYSTEMS AND METHODS FOR MITIGATING TISSUE BREAKDOWN,” the entirety of which is incorporated by reference herein.

### TECHNICAL FIELD

The present specification generally relates to systems and methods for determining changes in tissue status, and more specifically, systems and methods for mitigating tissue breakdown using networked sensors.

### BACKGROUND

Tissue breakdown or changes in tissue status may be early indicators of issues such as wound infection, inflammation, wound chronicity, or pressure ulcers. For example, pressure ulcers tend to develop in bedridden individuals with limited mobility. Conventionally, detection of such pressure ulcers depends on visual detection by a caregiver. A “stage 1” pressure ulcer may be identified when a caregiver applies pressure to the individual's skin and the skin does not turn white. Such detection methods do not provide sufficient advanced notice before a pressure ulcer develops and are subject to caregiver observation, which may vary from caregiver to caregiver.

As another example, biomarkers may be sensed to determine tissue status generally, and may be used to identify a chronic wound or infection. However, such biomarkers may vary greatly from individual to individual, making identification of tissue breakdown difficult.

Accordingly, a need exists for alternative systems and methods which provide early indication of a change in tissue status.

### SUMMARY

According to some embodiments of the present disclosure, a method for tracking a change in tissue status includes receiving at least some demographic information regarding an individual being monitored, receiving information regarding a first tissue status for the individual, comparing the received information with a threshold tissue status selected at least in part based on the at least some demographic information regarding the individual, and providing an alert indicative of a change in tissue status in response to determining that the received information exceeds the threshold tissue status.

According to some embodiments of the present disclosure, a system includes at least one sensor, a server, and at least one computing device. The sensor senses information regarding a tissue status for an individual. The server is communicatively coupled to the at least one sensor for receiving the information regarding the tissue status for the individual from the at least one sensor. The server includes a processor and a memory storing computer readable and executable instructions that, when executed by the processor, cause the server to compare the information regarding the tissue status to a threshold tissue status and provide at least one output based on the comparison. The at least one computing device is communicatively coupled to the server for receiving the at least one output.

Additional features and advantages will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

Referring now to the illustrative examples in the drawings, wherein like numerals represent the same or similar elements throughout:

FIG. 1 is a schematic illustrating an example system for mitigating tissue breakdown in accordance with one or more embodiments; and

FIG. 2 is a flow chart illustrating an example method for mitigating tissue breakdown in accordance with one or more embodiments.

### DETAILED DESCRIPTION

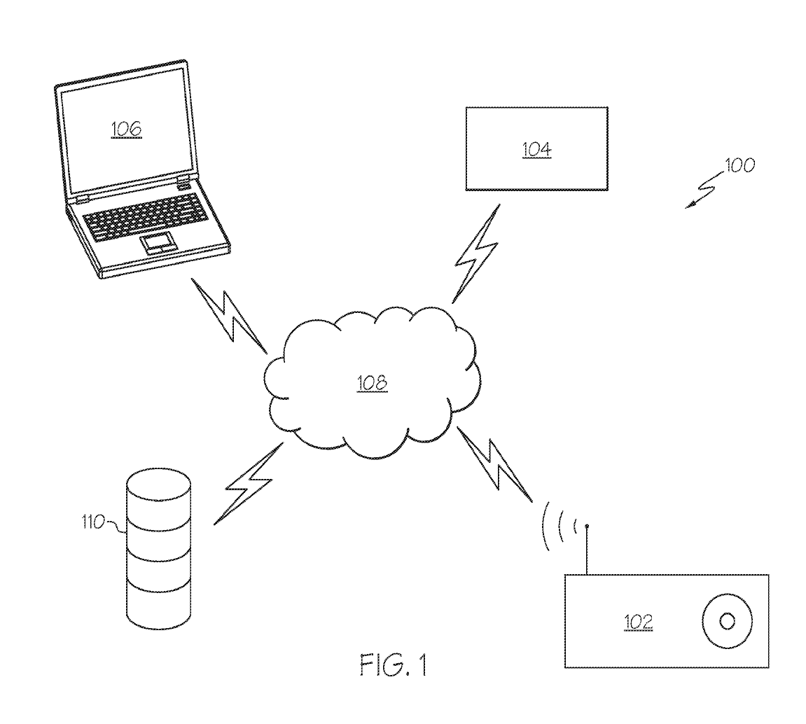

FIG. 1 generally depicts one embodiment of a system for mitigating tissue breakdown. The system generally includes a sensor, a server, and a computing device communicatively coupled via a network. Through the network, the server receives at least some demographic information regarding an individual being monitored; receives, from the sensor, information regarding a tissue status for the individual; compares the received information with a threshold tissue status selected at least in part based on the demographic information; and responsive to determining that the received information exceed the threshold tissue status, provides an alert indicative of a change in tissue status. A change in tissue status may be indicative of, for example, a pressure ulcer, a wound infection, inflammation, or wound chronicity. Various embodiments of the system and methods implemented by the system will be described in more detail herein.

A system 100 according to various embodiments is depicted in FIG. 1. As shown in FIG. 1, the system 100 includes a sensor 102, a server 104 communicatively coupled to the sensor 102, and a computing device 106. The computing device 106 is communicatively coupled to the server 104 and the sensor 102 via a network 108. In various embodiments, the system 100 may also include one or more databases 110 which may also be communicatively coupled via the network. However, in some embodiments, the one or more databases 110 may be incorporated into server 104.

In various embodiments, the sensor 102 is configured to sense and/or collect information regarding tissue. Such information collected by the sensor 102 may include, by way of example and not limitation, a temperature of the tissue, a moisture level of the tissue, information regarding mechanical energy dissipation through the tissue, information obtained via ultrasound or other imaging techniques, light frequency or spectral information, or information regarding a biomarker in the tissue. Accordingly, the sensor 102 may be implemented as a multiple audible or visible wavelength sensor, a light source coupled with an optical detector, a temperature sensor, a moisture sensor, an ultrasound or other high frequency, subsurface imaging sensor, a vibration sensor, an electrical sensor, or a thin-film circuit sensor operable to measure capacitance. The system 100 may include more than one sensor 102, and in such embodiments, the sensors 102 may be of varying types and be placed in various locations on the individual's body.

In some embodiments, the sensor 102 may be incorporated into a dressing or may otherwise be adhered to the individual. For example, the sensor 102 may include a thin patch of polyester with an adhesive that secures the sensor to the skin of the individual. The sensor 102 may also include a thin-film circuit mounted to the polyester patch. The thin-film circuit may include wireless communication circuitry and sensor circuitry. In some embodiments, the sensor 102 may be a passive, unpowered device that is operable to be energized inductively. However, in other embodiments, the sensor may include a charge source, such as a battery, to power the circuitry.

In embodiments in which the sensor 102 detects biomarkers, sensor circuitry may include metal oxide-type semi-conductor sensors and surface acoustic wave devices. In some embodiments, binding proteins, receptors, and/or enzymes may be used in addition to electrochemical transducers to generate an electrical signal in response to detection of a chemical. Sensor circuitry may, additionally or alternatively, include other components depending on the sensor employed and the information to be collected.

According to various embodiments, sensor 102 is a wireless sensor operable to transmit the information it collects to the server 104 via the network 108. In such embodiments, the sensor 102 may include wireless communication circuitry. In some embodiments, the sensor 102 may communicate via the network 108 directly, but in other embodiments, the sensor 102 may transmit the information through a patient support apparatus in close proximity to the sensor 102. For example, the wireless communication circuitry may include short range communications components that enable the sensor 102 to communicate with a receiver in the patient support apparatus via Bluetooth, RFID, or the like. In such embodiments, the person support apparatus may be connected to the network 108 and may transmit the information to the server 104 via the network 108.

In various embodiments, the server 104 generally includes one or more processors, memory, and network communication circuitry. The server 104 can provide various resources to the sensor 102 and/or the computing device 106 via the network. Resources can include providing, for example, processing, storage, software, and information from the server to the sensor 102 and/or the computing device 106. In various embodiments, the server 104 may be part of a server farm or cloud of servers which share resources with one another via wired portions of the network, wireless portions of the network, or combinations thereof.

In various embodiments, the server 104 provides capability to recognize changes in tissue status. For example, the server 104 can include one or more algorithms capable of identifying a change in tissue status and providing an output regarding that change in tissue status. More specifically, the server 104 can include a memory that stores computer readable and executable instructions that, when executed by the processor, cause the server to compare information regarding the tissue status to a threshold tissue status and provide at least one output based on the comparison. As will be described in greater detail hereinbelow, the output may be provided to the sensor 102 or to the computing device 106 via the network 108.

The computing device 106 may be any device having hardware (e.g., chipsets, processors, memory, etc.) for communicatively coupling with the network 108. Specifically, the computing device may be a mobile device, a desktop computing device, or a computing device incorporated into or attached to the person support apparatus, depending on the particular embodiment. For example, the computing device 106 may be a smart phone, a tablet device, an e-reader, a laptop computer, a desktop computer, or a computer associated with the person support apparatus. In various embodiments, the computing device 106 may be a device accessible by one or more caregivers, such as a computing device located at a nurses' station, in a doctor's office, or carried by the caregiver. Accordingly, in various embodiments, the computing device 106 receives an alert from the server 104 via the network 108. The alert can be, for example, a notification that the individual may be developing one or more issues associated with a change in tissue status, an instruction to make one or more adjustments to the individual or the person support apparatus, to administer one or more medications, to change a dressing, or to take some other action.

In various embodiments, the computing device 106 includes one or more memory components, one or more processing devices, a display, a speaker, at least one input device, and network interface hardware. The at least one input device can include, by way of example and not limitation, a microphone, a keyboard, a touch screen, a mouse, or the like. The network interface hardware may depend on the particular embodiment, and may include the hardware to enable the computing device 106 to communicate via network 108. The display can include any medium capable of transmitting an optical output such as, for example, a cathode ray tube, light emitting diodes, a liquid crystal display, a plasma display, or the like. Moreover, in some embodiments, the display is a touchscreen that, in addition to providing optical information, detects the presence and location of a tactile input upon a surface of or adjacent to the display. The computing device 106 may include additional or fewer components, depending on the particular embodiment. For example, in embodiments in which the computing device 106 is a smart phone, it may further include cellular network hardware and a microphone and not include a mouse, while in embodiments in which the computing device 106 is a desktop computing device, it may include a keyboard and a mouse and not include a touch screen.

As noted hereinabove, the computing device 106 receives an alert from the server 104. The alert may be, for example, an audible alert, a visual alert, a vibratory alert, or a combination of these types of alerts. For example, in various embodiments, a notification may pop up on the display of the computing device 106 while in other embodiments, a sound may play to further alert the caregiver to the presence of the notification.

In addition to receiving an alert from the server 104, the computing device 106 may receive a graphical history of the tissue status over time for the individual. For example, in some embodiments, the server 104 may provide the graphical history which is displayed by the computing device 106. The graphical history may illustrate one or more types of information collected by the sensor 102 over time. In some embodiments, a user viewing the graphical history can adjust the view, zoom in or out to view more or fewer points in time, and/or select one or more options related to the historical view. As but one example, a user may change the view from a line graph of the temperature of the tissue over time to an image generated by a subsurface imaging device. Once viewing the image generated by the subsurface imaging device, the user may select between images collected on any particular day, select a side-by-side view of one or more images taken at different times, or the like.

In various embodiments, an alert or other output is generated by the server 104 responsive to comparing received information regarding a tissue status with a threshold tissue status and determining that the received information exceeds the threshold status. The threshold tissue status may be, by way of example and not limitation, a temperature value, a moisture level, an impedance, a presence or absence of a biomarker, a concentration of a biomarker, a spectrum, a rate of change of a measured value, a light intensity at a predetermined wavelength, or the like.

In various embodiments, the threshold is quantified as a threshold value, which may be, for example, a measured value (such as temperature, moisture, impedance, rate of change, etc.) or a value extracted from one or more images or other collected information. Threshold values may be stored in memory or a database associated with the server 104. However, in some embodiments, such as the embodiment illustrated in FIG. 1, the database 110 may be located remotely from the server 104.

Regardless of its location relative to the server 104, the database 110 may include one or more signature libraries, historical information for one or more individuals, demographic information for one or more individuals, and threshold values, among other information. Using the information in database 110, the server 104 can determine whether the information received from the sensor 102 represents a change in tissue status for the individual, whether the change in tissue status exceeds a threshold, and/or whether the tissue status is indicative of an issue such as a pressure ulcer, a wound infection, inflammation, or wound chronicity. For example, a tissue temperature above a particular threshold temperature or rising at a particular rate of change may indicate that the tissue is warming, and may be an indication of early wound infection or early stage breakdown of the tissue. As another example, a moisture level measured below the epidermis that exceeds a particular moisture level may indicate early inflammation, which may result in a stage I pressure ulcer. Similarly, ultrasound or other high-frequency subsurface imaging techniques may indicate the presence of a pocket of fluid indicative of inflammation, delamination of the dermal/epidermal junction indicative of shear damage, or the existence of another subsurface tissue injury that may worsen over time.

In embodiments in which the sensor is a light source and an optical detector, a change in light intensity at a predetermined wavelength may be indicative of early tissue breakdown. For example, a spectral change or change in light intensity at a certain wavelength due to the presence of biomarkers in tissue may be detected.

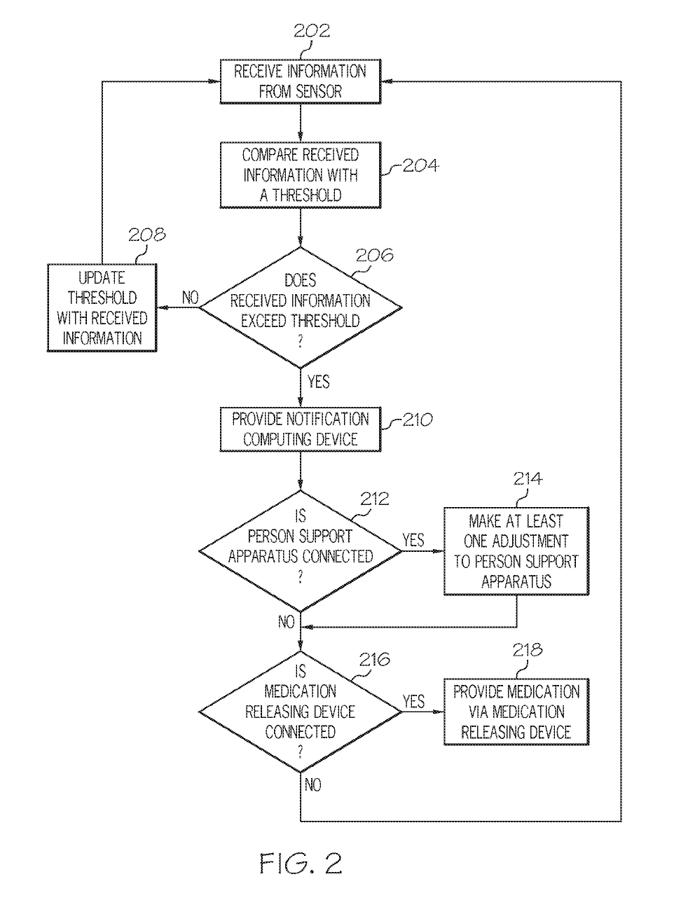

Turning now to FIG. 2, an example process of mitigating tissue breakdown is illustrated. The process may be implemented, for example, by the server 104. However, in some embodiments, one or more of the steps in the process may be implemented by the sensor 102 and/or the computing device 106.

At block 202, the server receives information from the sensor. The information received may depend on the particular sensor employed. For example, a temperature sensor may transmit a value or a signal corresponding to a temperature of the tissue, while an optical sensor may transmit a signal corresponding to an image of the tissue. The server then compares the received information with a threshold (block 204). As described hereinabove, in some embodiments, the threshold may be selected based on one or more pieces of information corresponding to the individual, such as one or more pieces of demographic information.

At block 206, the server determines whether the received information exceeds the threshold. In various embodiments, the threshold is a quantified value that can be compared to a quantified value related to the received information. As used herein, the phrase “exceeds the threshold” includes instances where the value related to the received information is greater than the quantified threshold value, where the quantified threshold value is a minimum value indicative of a condition, as well as instances where the value related to the received information is less than the quantified threshold value, where the quantified threshold value is a maximum value indicative of a condition.

In various embodiments, the threshold to which information is compared is a dynamic threshold. For example, the threshold may vary depending on one or more pieces of demographic information for the individual, and/or may be updated over time to reflect additional data. Demographic information for the individual may be provided via one or more computing devices connected to the network 108. For example, a caregiver may enter patient information at a computing device 106 located at a nurses' station to enable the system to associate changes in the tissue status with the particular individual. Information may be provided, for example, in electronic profiles, electronic files, or the like, and may be stored in a database to be accessed by the server 104.

Demographic information for the individual can include an age of the individual, a medical condition of the individual, a sex of the individual, a weight of the individual, a medication provided to the individual, a smoking status of the individual, a mobility level of the individual, a medical device utilized by the individual, or an indication that the individual has previously had the tissue condition. Medical conditions that may impact the threshold can include pneumonia, sepsis, a bacterial infection and anemia, among others. In various embodiments, the individual can be associated with one or more demographics based on his or her demographic information and the received information regarding tissue status may be compared to a threshold based at least in part on the one or more demographics with which the individual is associated.

As but one example, when an individual enters a care facility, a caregiver may create a profile for the individual that indicates that the individual is a 75 year old African-American woman. The caregiver may further indicate in the individual's profile that the individual has not previously had pressure ulcers, has low mobility, and logs the individual's weight. When the sensor 102 collects information regarding a tissue status for this individual, the collected information may be associated with the individual's profile. Additionally, when the server 104 compares the collected information with a threshold, the threshold may be a threshold calculated for women over the age of 70 with low mobility and no previous history of pressure ulcers.

It should be understood that threshold may be selected based on one or more pieces of demographic information, depending on the demographic information collected, the processing power, and the level of confidence associated with thresholds for particular demographics. For example, where there is only a single individual in the system that is under age 60, the age of the individual may be disregarded because there is not a sample size large enough to give a threshold value that has an acceptable confidence value.

In addition to enabling the server 104 to select an appropriate threshold value for comparison to received information, the demographic information and/or the information received from the sensor may additionally be used to further adjust the thresholds. In various embodiments, the server 104 includes a machine-learning algorithm that dynamically adjusts the threshold based on the received tissue status information. For example, returning to FIG. 2, when the received information does not exceed the threshold (i.e., a “no” at block 206), in various embodiments, the server updates the threshold with the received information (block 208) and waits for the sensor to transmit additional information.

Consider the following example. An individual enters a care facility, a caregiver creates a profile for the individual, and one or more sensors 102 are positioned to collect or sense information regarding a tissue status for the individual. In this example, the sensor collects information regarding subepidermal moisture (SEM) levels as a measure of impedance in dermal phase units (DPU), although other values may be collected depending on the particular embodiment. The information is transmitted to the server 104, which receives the information and compares the received information with a threshold tissue status that is based on at least some demographic information for the individual (e.g., one or more pieces of information in the individual's profile). During the first three days, the individual's levels are within the “normal” range, or do not exceed the threshold values. For example, during the first three days, the individual's SEM is 104 DMU, 120 DMU, and 130 DMU, respectively. However, on the fourth day, the individual's SEM is approximately 185 DMU, which exceeds the threshold value of 150 DMU. Therefore, the server 104 provides an alert indicative of a change in tissue status. In addition, the server 104 updates the threshold. For example, if the threshold of 150 DMU was based on an average SEM for 20 patients within the individual's demographic that developed pressure ulcers within one week, the threshold value may be recalculated to include the individual's SEM of 185 DMU, adjusting the threshold value up or down, depending on the other values within the demographic.

In some embodiments, the threshold is updated based on an outcome. For example, the values for an individual may be logged but not utilized to adjust an applicable threshold until after a final determination regarding a tissue status has been made, such as the development of a pressure ulcer or the absence of a pressure ulcer. In such embodiments, the dynamic threshold may be adjusted depending on the final outcome for the patient.

In various embodiments, the server 104 makes one or more automated decisions based on the comparison of the collected information with data in the database. The automated decisions may include, by way of example and not illustration, making an adjustment to a person support apparatus upon which the individual is disposed, providing a notification to a caregiver, or providing medication to the individual.

Returning to FIG. 2, responsive to determining that the received information exceeds the threshold (i.e., a “yes” at block 206), the server provides a notification to one or more computing devices (block 210). In some embodiments, such as the embodiment illustrated in FIG. 2, after providing the notification, the server additionally determines if a person support apparatus is connected at block 212. For example, the server may determine whether a controller associated with the person support apparatus upon which the individual is disposed is connected via the network. If a person support apparatus is connected (i.e., a “yes” at block 212), at block 214, the server makes at least one adjustment to the person support apparatus. For example, the server may transmit an instruction to the person support apparatus to alter an angle of the person support apparatus, an instruction to reduce a pressure associated with the person support apparatus, or an instruction to alter a temperature of the person support apparatus. When received by the person support apparatus, the person support apparatus may make the adjustment as instructed. For example, the server may provide an instruction to decrease a pressure associated with the person support apparatus and the controller may cause the pressure of the fluidized mattress of the person support apparatus to be decreased. It should be understood that the instructions provided to the person support apparatus may vary depending on the particular embodiment, and may depend on the features of the person support apparatus employed.

In various embodiments, when the server determines that a person support apparatus is not connected (i.e., a “no” at block 212), the server determines whether a medication releasing device is connected at block 216. For example, the server may determine whether an IV, bandage or dressing positioned on the individual is capable of automatically providing one or more medications and is connected via the network. In various embodiments, the server determines whether a medication releasing device is connected after making at least one adjustment to the person support apparatus at block 214.

When the server determines that a medication releasing device is connected (i.e., a “yes” at block 216), the server provides medication via the medication releasing device. For example, the server may transmit an instruction to the person support apparatus to administer a predetermined dosage of medication to the individual. It should be understood that the instructions regarding the medication to be administered and the dosage may vary depending on the particular embodiment and the condition determined based on the received information.

If the server determines that a medication releasing device is not connected (i.e., a “no” at block 216), the server returns to the beginning of the process and waits for the sensor to transmit additional information.

It should be understood that the process set forth in FIG. 2 is exemplary. Thus, it is contemplated that various embodiments may include all or some of the process steps shown in FIG. 2. For example, some embodiments may include various automated decisions, or combinations thereof. Some examples may include steps of providing a notification to a computing device and determining if a medication releasing device is connected, but not include the step of determining if a person support apparatus is connected, while other embodiments may include steps of providing a notification to a computing device and determining if a person support apparatus is connected, but not include determining if a medication releasing device is connected. Still other embodiments may not include providing a notification to a computing device. Additionally, some embodiments may include other automated decisions based on the comparison of the received data to the threshold that are effective to reduce the development of a pressure ulcer or other tissue breakdown.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

What is claimed is:

1. A method for tracking a change in tissue status comprising:
receiving at least some demographic information regarding an individual being monitored;
receiving, from a sensor, information regarding a first tissue status for the individual;
comparing the received information with a threshold tissue status selected at least in part based on the at least some demographic information regarding the individual;
providing an alert indicative of a change in tissue status in response to determining that the received information exceeds the threshold tissue status.

2. The method of claim 1, wherein the at least some demographic information regarding the individual being monitored comprises at least one of an age of the individual, a medical condition of the individual, a sex of the individual, a weight of the individual, a medication provided to the individual, a smoking status of the individual, a mobility level of the individual, a medical device utilized by the individual, and an indication that the individual has previously had a tissue condition.

3. The method of claim 1, wherein the information regarding the first tissue status for the individual comprises a temperature of the tissue, a moisture level of the tissue, mechanical energy dissipation through the tissue, ultrasound or other high frequency information, light frequency information, or a biomarker in the tissue.

4. The method of claim 1, wherein providing the alert indicative of the change in tissue status comprises providing an alert to a caregiver via a computing device.

5. The method of claim 1, further comprising:
storing the received information regarding the first tissue status and the at least some information regarding an individual being monitored in a database;
receiving, from the sensor, information regarding at least one subsequent tissue status for the individual; and
updating the database with the at least one subsequent tissue status for the individual.

6. The method of claim 5, further comprising:
adjusting the threshold based on at least some of the information in the database.

7. The method of claim 5, further comprising providing a graphical history of tissue status over time based on the first tissue status and the at least one subsequent tissue status for the individual.

8. The method of claim 5, further comprising updating the database with an outcome for the individual.

9. The method of claim 1, further comprising making an adjustment to a person support apparatus upon which the individual is disposed.

10. A system comprising:
at least one sensor for sensing information regarding a tissue status for an individual;
a server communicatively coupled to the at least one sensor for receiving the information regarding the tissue status for the individual from the at least one sensor, the server comprising a processor and a memory storing computer readable and executable instructions that, when executed by the processor, cause the server to compare the information regarding the tissue status to a threshold tissue status and provide at least one output based on the comparison; and
at least one computing device communicatively coupled to the server for receiving the at least one output from the server.

11. The system of claim 10, wherein the sensor comprises a light source and an optical detector.

12. The system of claim 10, wherein the information regarding the tissue status for the individual comprises information regarding a change in light intensity at a predetermined wavelength.

13. The system of claim 12, wherein the at least one output comprises an instruction to make an adjustment to a person support apparatus upon which the individual is disposed, a notification to a caregiver, or an instruction to provide medication.

14. The system of claim 13, wherein the instruction to make the adjustment to the person support apparatus comprises an instruction to alter an angle of the person support apparatus, an instruction to reduce a pressure associated with the person support apparatus, or an instruction to alter a temperature of the person support apparatus.

15. The system of claim 12, wherein the at least one computing device is associated with a person support apparatus upon which the individual is disposed.

16. The system of claim 10, wherein the server receives at least some information regarding the individual, and the threshold tissue status depends at least in part on the at least some information regarding the individual.

17. The system of claim 16, wherein the server updates the threshold tissue status based on the information regarding the tissue status for the individual.

18. The system of claim 16, wherein the server updates the threshold tissue status based on an outcome for the individual.

19. The system of claim 16, wherein the at least some information regarding the individual comprises at least one of an age of the individual, a medical condition of the individual, a sex of the individual, a weight of the individual, a medication provided to the individual, a smoking status of the individual, a mobility level of the individual, a medical device utilized by the individual, and an indication that the individual has previously had a tissue condition.

20. The system of claim 10, wherein the information regarding the tissue status comprises information regarding a moisture level of the tissue, mechanical energy dissipation through the tissue, ultrasound or other high frequency information, light frequency information, or a biomarker in the tissue.
